(19) United States
(12) Patent Application Publication
(10) Pub. No.: US 2017/0128426 A1
(43) Pub. Date: May 11, 2017

(54) NOVEL DOSAGE AND FORMULATION

(71) Applicant: Almirall, S.A., Barcelona (ES)

(72) Inventors: Rosa LAMARCA CASADO, Barcelona (ES); Gonzalo DE MIQUEL SERRA, Barcelona (ES)

(21) Appl. No.: 15/409,741

(22) Filed: Jan. 19, 2017

Related U.S. Application Data
(63) Continuation of application No. 14/403,220, filed on Nov. 24, 2014, now abandoned.
(60) Provisional application No. 61/779,578, filed on Mar. 13, 2013.
(60) Provisional application No. 61/654,224, filed on Jun. 1, 2012.

(30) Foreign Application Priority Data
May 25, 2012 (EP) 12382211.6

(51) Int. Cl. A61K 31/439; A61M 15/00; A61K 31/167; A61K 47/26; A61K 9/00; A61K 45/06

(52) U.S. Cl. CPC A61K 31/439; A61K 9/0075; A61K 45/06; A61K 31/167; A61K 47/26; A61M 15/0086; A61M 15/0038; A61M 2202/064

(57) ABSTRACT

A pharmaceutical composition for inhalation comprising aclidinium in the form of a dry powder of a pharmaceutically acceptable salt in admixture with a pharmaceutically acceptable dry powder carrier, providing a delivered dose of aclidinium equivalent to about 322 micrograms aclidinium free base.

This invention relates to a novel dosage for aclidinium and to novel methods and formulations for the treatment of respiratory diseases, especially asthma and chronic obstructive pulmonary disease (COPD), using aclidinium.

### BACKGROUND

Aclidinium bromide is 3(R)-(2-hydroxy-2,2-dithien-2-ylacetoxy)-1-(3-phenoxypropyl)-1-azoniabicyclo[2.2.2]octane bromide, described in, e.g., WO 01/04118. An optimized process for the production of aclidinium bromide is described in WO 2008/009397. The structural formula is

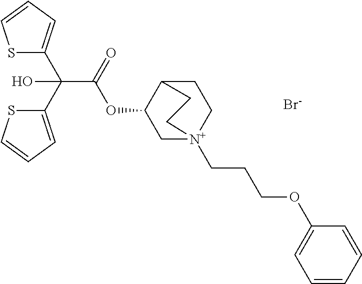 [SMILES: O=C(O[C@H]1C[N+]2(CCCOc3ccccc3)CCC1CC2)C(O)(c1cccs1)c1cccs1 ; [Br-]]

Aclidinium bromide is a white powder with a molecular formula of C₂₆H₃₀NO₄S₂Br and a molecular mass of 564.56. It is very slightly soluble in water and ethanol and sparingly soluble in methanol. This compound is known to be a long-acting anticholinergic useful in the treatment of respiratory diseases.

### SUMMARY OF THE INVENTION

It is now surprisingly found that, for treatment of respiratory disorders, particularly asthma and COPD, in an adult human, aclidinium is most effective upon administration by inhalation in a dosage of about 322 micrograms (μg) delivered dose (weight corresponding to aclidinium free base, i.e. free ammonium cation), and/or a fine particle dose equivalent to about 140 μg aclidinium bromide. Typically the dose is a single dose or a twice daily dose, preferably a twice daily dose.

Typically, a delivered dose of about 322 μg aclidinium free base corresponds to a delivered dose of about 375 μg aclidinium bromide.

The invention thus provides in a first embodiment a pharmaceutical composition for inhalation comprising aclidinium in the form of a dry powder of a pharmaceutically acceptable salt, e.g., aclidinium bromide, in admixture with lactose powder (i.e. lactose particles), (i) providing a delivered dose of aclidinium equivalent to about 322 μg aclidinium (per inhalation) and/or a fine particle dose equivalent to about 140 μg aclidinium bromide (per inhalation), or (ii) in a multidose dry powder inhaler device calibrated to provide a delivered dose of aclidinium equivalent to about 322 μg aclidinium (per inhalation) and/or a fine particle dose equivalent to about 140 μg aclidinium bromide (per inhalation). This composition can be administered one or more times per day. Preferably once or twice a day.

In a second embodiment, the invention provides a method of treating a respiratory condition, e.g., selected from asthma and chronic obstructive pulmonary disease, in a patient in need of such treatment, comprising administering a dose, typically a single daily dose or twice daily dose, of aclidinium, e.g., aclidinium bromide, equivalent to about 322 μg aclidinium and/or a fine particle dose equivalent to about 140 μg aclidinium bromide, e.g., comprising administering a pharmaceutical composition according to the previous paragraph. The invention further provides the use of aclidinium in the manufacture of a medicament, e.g., as described in the preceding paragraph, for use in such a method.

The aclidinium may be administered as monotherapy, or in combination with one or more additional anti-inflammatory and/or bronchodilating agents, e.g., corticosteroids, PDE IV inhibitors and β2-agonists, e.g., formoterol, salmeterol, budesonide, and mometasone, and the invention thus further provides methods as described above further comprising administration of an effective amount of such an agent, as well as pharmaceutical compositions as described above, further comprising such additional agent(s).

### DETAILED DESCRIPTION OF THE INVENTION

Typically, aclidinium is administered in the form of a salt with an anion X, wherein X is a pharmaceutically acceptable anion of a mono or polyvalent acid. More typically, X is an anion derived from an inorganic acid, such as hydrochloric acid, hydrobromic acid, sulphuric acid and phosphoric acid, or an organic acid such as methanesulphonic acid, acetic acid, fumaric acid, succinic acid, lactic acid, citric acid or maleic acid. Preferably aclidinium is in the form of aclidinium bromide.

The aclidinium is preferably administered in the form of a dry powder, in admixture with a suitable carrier, e.g., lactose powder (i.e. lactose particles), suitable for inhalation. In a preferred embodiment, the lactose is in the form of alpha-lactose monohydrate, preferably crystalline alpha-lactose monohydrate.

For example, in one embodiment, the aclidinium is aclidinium bromide in admixture with lactose powder.

The respiratory disease or condition to be treated with the formulations and methods of the present invention is typically asthma, acute or chronic bronchitis, emphysema, chronic obstructive pulmonary disease (COPD), bronchial hyperreactivity or rhinitis, in particular asthma or chronic obstructive pulmonary disease (COPD), especially COPD.

For the avoidance of doubt, by delivered dose it is meant the amount of the drug which is available at the mouth for inhalation (dose emitted from the mouthpiece of the inhaler device per actuation). The delivered dose can be measured using standard techniques known to those skilled in the art. In the context of dosage of an active agent, “about” as used herein means within the normal limits of acceptable variations as defined by the European and US Pharmacopeia of plus/minus 35% or preferably acceptable variations as defined by the current most stringent requirement, the US FDA draft guidance for inhaler of plus/minus 25%, or more preferably according to the CHMP Guideline on the Pharmaceutical Quality of Inhalation and Nasal Products of plus/minus 15%, and especially within the metered dosing accuracy for the dispensing system e.g. plus/minus 10%. Thus, by a delivered dose of “about 322 μg aclidinium free base” it is meant a target dose of 322 μg aclidinium subject to variation within the normal limits of acceptance for the dispensing system, e.g. 209-435 μg aclidinium (plus/minus 35%, acceptable variations as defined by the European and US Pharmacopeia) or preferably 241-403 μg aclidinium (plus/minus 25%, acceptable variations as defined by the current most stringent requirement, the US FDA draft guidance for inhaler), or more preferably 273-371 μg aclidinium (plus/minus 15%, acceptable variations defined by the CHMP Guideline on the Pharmaceutical Quality of Inhalation and Nasal Products) or especially 289-355 μg aclidinium (or within the metered dosing accuracy of the Inhaler).

By a delivered dose of “about 375 μg aclidinium bromide” it is meant a target dose of 375 μg aclidinium bromide subject to variation within the normal limits of acceptance for the dispensing system, e.g. 242-507 μg aclidinium bromide (plus/minus 35%, acceptable variations as defined by the European and US Pharmacopeia) or preferably 281-469 μg aclidinium bromide (plus/minus 25%, acceptable variations as defined by the current most stringent requirement, the US FDA draft guidance for inhaler), or more preferably 319-431 μg aclidinium bromide (plus/minus 15%, acceptable variations defined by the CHMP Guideline on the Pharmaceutical Quality of Inhalation and Nasal Products) or especially 337-413 μg aclidinium bromide (or within the metered dosing accuracy of the inhaler).

The fine particle dose (fine particle dose=μg aclidinium/aclidinium bromide in the delivered dose below a cut off aerodynamic threshold of 5 micrometer) are also subjected to variations. Therefore, by a fine particle dose of “about 140 μg aclidinium bromide” it is meant a target dose of 79-206 μg aclidinium bromide, preferably 100-190 μg aclidinium bromide, more preferably 110-180 μg aclidinium bromide. The fine particle dose can be measured using standard techniques known to those skilled in the art. Typically, a fine particle dose of 140 μg aclidinium bromide corresponds to a fine particle dose of about 120 μg aclidinium. By a fine particle dose of “about 120 μg aclidinium” it is meant a target dose of 67-139 μg aclidinium, preferably 86-163 μg aclidinium, more preferably 94-155 μg aclidinium.

In a preferred embodiment, the invention is direct to a pharmaceutical composition for inhalation comprising aclidinium, in the form of a dry powder of a pharmaceutically acceptable salt, i.e. aclidinium bromide, in admixture with lactose powder (i.e. alpha-lactose monohydrate lactose particles), providing a fine particle dose equivalent to about 120 μg aclidinium (aclidinium free ammonium cation), which corresponds to about 140 μg aclidinium bromide per inhalation, preferably 86-163 μg aclidinium (aclidinium free ammonium cation), which corresponds to 100-190 μg aclidinium bromide per inhalation. Typically the dose is a single dose or a twice daily dose, preferably a twice daily dose.

Packaging of the formulation may be suitable for unit dose or multi-dose delivery. In the case of multi-dose delivery, the formulation can be pre-metered or metered in use. Dry powder inhalers are thus classified into three groups: (a) single dose, (b) multiple unit dose and (c) multi dose devices.

Formulations generally contain a powder mix for inhalation of the compounds of the invention and a suitable powder base (carrier substance) such as lactose. Each capsule or cartridge may generally contain between 2 μg and 400 μg of each therapeutically active ingredient. Alternatively, the active ingredient (s) may be presented without excipients.

For single dose inhalers of the first type, single doses have been weighed by the manufacturer into small containers, which are mostly hard gelatine capsules. A capsule has to be taken from a separate box or container and inserted into a receptacle area of the inhaler. Next, the capsule has to be opened or perforated with pins or cutting blades in order to allow part of the inspiratory air stream to pass through the capsule for powder entrainment or to discharge the powder from the capsule through these perforations by means of centrifugal force during inhalation. After inhalation, the emptied capsule has to be removed from the inhaler again. Mostly, disassembling of the inhaler is necessary for inserting and removing the capsule, which is an operation that can be difficult and burdensome for some patients. Other drawbacks related to the use of hard gelatine capsules for inhalation powders are (a) poor protection against moisture uptake from the ambient air, (b) problems with opening or perforation after the capsules have been exposed previously to extreme relative humidity, which causes fragmentation or indenture, and (c) possible inhalation of capsule fragments. Moreover, for a number of capsule inhalers, incomplete expulsion has been reported.

Some capsule inhalers have a magazine from which individual capsules can be transferred to a receiving chamber, in which perforation and emptying takes place, as described in WO 92/03175. Other capsule inhalers have revolving magazines with capsule chambers that can be brought in line with the air conduit for dose discharge (e. g. WO91/02558 and GB 2242134). They comprise the type of multiple unit dose inhalers together with blister inhalers, which have a limited number of unit doses in supply on a disk or on a strip.

Blister inhalers provide better moisture protection of the medicament than capsule inhalers. Access to the powder is obtained by perforating the cover as well as the blister foil, or by peeling off the cover foil. When a blister strip is used instead of a disk, the number of doses can be increased, but it is inconvenient for the patient to replace an empty strip. Therefore, such devices are often disposable with the incorporated dose system, including the technique used to transport the strip and open the blister pockets.

Multi-dose inhalers do not contain pre-measured quantities of the powder formulation. They consist of a relatively large container and a dose measuring principle that has to be operated by the patient. The container bears multiple doses that are isolated individually from the bulk of powder by volumetric displacement. Various dose measuring principles exist, including rotatable membranes (e. g. EP0069715) or disks (e. g. GB 2041763; EP 0424790; DE 4239402 and EP 0674533), rotatable cylinders (e. g. EP 0166294; GB 2165159 and WO 92/09322) and rotatable frustums (e. g. WO 92/00771), all having cavities which have to be filled with powder from the container. Other multi dose devices have measuring slides (e. g. U.S. Pat. No. 5,201,308 and WO 97/00703) or measuring plungers with a local or circumferential recess to displace a certain volume of powder from the container to a delivery chamber or an air conduit e. g. EP 0505321, WO 92/04068 and WO 92/04928.

Reproducible dose measuring is one of the major concerns for multi dose inhaler devices. The powder formulation has to exhibit good and stable flow properties, because filling of the dose measuring cups or cavities is mostly under the influence of the force of gravity. For reloaded single dose and multiple unit dose inhalers, the dose measuring accuracy and reproducibility can be guaranteed by the manufacturer. Multi dose inhalers on the other hand, can contain a much higher number of doses, whereas the number of handlings to prime a dose is generally lower.

Because the inspiratory air stream in multi-dose devices is often straight across the dose measuring cavity, and because the massive and rigid dose measuring systems of multi dose inhalers cannot be agitated by this inspiratory air stream, the powder mass is simply entrained from the cavity and little de-agglomeration is obtained during discharge.

Consequently, separate disintegration means are necessary. However in practice, they are not always part of the inhaler design. Because of the high number of doses in multi-dose devices, powder adhesion onto the inner walls of the air conduits and the de-agglomeration means must be minimized and/or regular cleaning of these parts must be possible, without affecting the residual doses in the device. Some multi dose inhalers have disposable drug containers that can be replaced after the prescribed number of doses has been taken (e. g. WO 97/000703). For such semi-permanent multi dose inhalers with disposable drug containers, the requirements to prevent drug accumulation are even stricter.

In a preferred embodiment, the aclidinium is administered via a breath-activated, multidose, dry powder inhaler, which delivers up to 200 metered doses from a non-removable cartridge. An especially preferred inhaler device for this purpose is Genuair®, (formerly known as Novolizer SD2FL), or as described in WO 97/00703, WO 03/000325 or WO 2006/008027 the contents of which applications are incorporated herein by reference. Genuair® is also described in H. Chrystyn et al. Int J Clin Pract, March 2012, 66, 3, 309-317; and in H. Magnussen et al. Respiratory Medicine (2009) 103, 1832-1837. Another breath-activated, multidose, dry powder inhaler suitable for the administration of aclidinium is Novolizer®, which is described in C. Fenton et al., Drugs 2003; 63 (22): 2437-2445; and D. Kohler, Respiratory Medicine (2004) Supplement A, S17-S21.

In another embodiment, aclidinium can also be administered via single dose dry powder inhalers such as the devices described in WO 2005/113042 or in EP1270034. These devices are low resistance unit dosage form inhalers. The unit dosage form of the dry powder formulation are capsules typically made of gelatin or a synthetic polymer, preferably hydroxypropyl methyl cellulose (HPMC), also known as hypromellose. The hypromellose capsules are preferably packaged in a blister. The blister is preferably a peel foil blister that allows patients to remove capsules stored therein without damaging them and optimizes product stability.

Apart from applications through dry powder inhalers the compositions of the invention can be administered in aerosols which operate via propellant gases or by means of so-called atomisers or nebulizers, via which solutions or suspensions of pharmacologically-active substances can be sprayed under high pressure so that a mist of inhalable particles results.

Medicaments for administration by inhalation desirably have a controlled particle size. The optimum particle size for inhalation into the bronchial system is usually 1-10 μm, preferably 2-5 μm. Particles having a size above 20 μm are generally too large when inhaled to reach the small airways. To achieve these particle sizes the particles of the active ingredient as produced may be size reduced by conventional means eg by micronisation or supercritical fluid techniques. The desired fraction may be separated out by air classification or sieving. Preferably, the particles will be crystalline.

Achieving a high dose reproducibility with micronised powders is difficult because of their poor flowability and extreme agglomeration tendency. To improve the efficiency of dry powder compositions, the particles should be large while in the inhaler, but small when discharged into the respiratory tract. Thus, an excipient, for example lactose is generally employed. The particle size of the excipient will usually be much greater than the inhaled medicament within the present invention. When the excipient is lactose it will typically be present as lactose particles, preferably crystalline alpha-lactose monohydrate, e.g., having an average particle size range of 20-1000 μm, preferably in the range of 90-150 μm. The median particle size approximately corresponds to the average and is the diameter where 50 mass-% of the particles have a larger equivalent diameter, and the other 50 mass-% have a smaller equivalent diameter. Hence the average particle size is generally referred to in the art as equivalent d50. The distribution of particle size around may affect flow properties, bulk density, etc. Hence to characterize a particle size diameter, other equivalent diameters can be used in addition to d50, such as d10 and d90. d10 is the equivalent diameter where 10 mass-% of the particles have a smaller diameter (and hence the remaining 90% is coarser). d90 is the equivalent diameter where 90 mass-% of the particles have a smaller diameter. In one embodiment, the lactose particles for use in formulations of the invention have a d10 of 90-160 μm, a d50 of 170-270 μm, and d90 of 290-400 μm.

Suitable lactose materials for use in the present invention are commercially available, e.g., from DMW Internacional (Respitose GR-001, Respitose SV-001, Respitose SV-003); Meggle (Capsulac 60, Inhalac 70, Capsulac 60 INH); and Borculo Domo (Lactohale 100-200, Lactohale 200-300, and Lactohale 100-300).

The ratio between the lactose particles and the aclidinium by weight will depend on the inhaler device used, but is typically, e.g., 5:1 to 100:1, for example 25:1 to 75:1, preferably 25:1 to 50:1, more preferably 30:1 to 35:1.

In a preferred embodiment, aclidinium is administered in the form of a dry powder formulation of aclidinium bromide in admixture with lactose, preferably alpha-lactose monohydrate, in a ratio by weight of aclidinium to lactose of 1:25 to 1:50, preferably 1:30 to 1:35, suitable for administration via a dry powder inhaler, wherein the aclidinium particles have an average particle size of from 2 to 5 μm in diameter, e.g., less than 3 μm in diameter, and the lactose particles have a d10 of 90-160 μm, a d50 of 170-270 μm, and d90 of 290-400 μm.

Additional active agents such as β2-agonists, PDE IV inhibitors, corticosteroids, leukotriene D4 antagonists, inhibitors of egfr-kinase, p38 kinase inhibitors or NK1 receptor agonists may be utilized in the methods and formulations of the inventions. For example, the invention provides aclidinium formulations as described herein further comprising an effective amount of one or more such additional active agents, e.g. further comprising an effective amount of a β2-agonist and/or a PDE IV inhibitor and/or a corticosteroid. The invention also provides methods for treating respiratory conditions as herein before described, e.g., asthma or COPD, comprising administering an aclidinium formulation as described herein and further comprising administering simultaneously an effective amount of one or more such additional active agents, e.g. further comprising an effective amount of a β2-agonist and/or a PDE IV inhibitor and/or a corticosteroid.

β2-agonists suitable for use with the aclidinium in the present invention include, e.g., arformoterol, bambuterol, bitolterol, broxaterol, carbuterol, clenbuterol, dopexamine, fenoterol, formoterol, hexoprenaline, ibuterol, isoetharine, isoprenaline, levosalbutamol, mabuterol, meluadrine, metaprotenerol, nolomirole, orciprenaline, pirbuterol, procaterol, reproterol, ritodrine, rimoterol, salbutamol, salmefamol, salmeterol, sibenadet, sotenerot, sulfonterol, terbutaline, tiaramide, tulobuterol, GSK-597901, milveterol, GSK-678007, GSK-642444, GSK-159802, HOKU-81, abediterol (LAS100977), KUL-1248, carmoterol, indacaterol and 5-[2-(5,6-diethylindan-2-ylamino)-1-hydroxyethyl]-8-hydroxy-1H-quinolin-2-one, 4-hydroxy-7-[2-{[2-{[3-(2-phenylethoxy)propyl]sulfonyl} ethyl]amino}ethyl]-2(3H)-benzothiazolone, 1-(2-fluoro-4-hydroxyphenyl)-2-[4-(1-benzimidazolyl)-2-methyl-2-butylamino]ethanol, 1-[3-(4-methoxybenzylamino)-4-hydroxyphenyl]-2-[4-(1-benzimidazolyl)-2-methyl-2-butylamino]ethanol, 1-[2H-5-hydroxy-3-oxo-4H-1,4-benzoxazin-8-yl]-2-[3-(4-N,N-dimethylaminophenyl)-2-methyl-2-propylamino]ethanol, 1-[2H-5-hydroxy-3-oxo-4H-1,4-benzoxazin-8-yl]-2-[3-(4-methoxyphenyl)-2-methyl-2-propylamino]ethanol, 1-[2H-5-hydroxy-3-oxo-4H-1,4-benzoxazin-8-yl]-2-[3-(4-n-butyloxyphenyl)-2-methyl-2-propylamino]ethanol, 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol, 5-hydroxy-8-(1-hydroxy-2-isopropylaminobutyl)-2H-1,4-benzoxazin-3-(4H)-one, 1-(4-amino-3-chloro-5-trifluoromethylphenyl)-2-tert-butylamino)ethanol and 1-(4-ethoxycarbonylamino-3-cyano-5-fluorophenyl)-2-(tert-butylamino)ethanol optionally in the form of their racemates, their enantiomers, their diastereomers, and mixtures thereof, and optionally their pharmacologically-compatible acid addition salts.

The preferred β2-agonists to be used in the combinations of the invention are: arformoterol, bambuterol, bitolterol, broxaterol, carbuterol, clenbuterol, dopexamine, fenoterol, formoterol, hexoprenaline, ibuterol, isoprenaline, levosalbutamol, mabuterol, meluadrine, nolomirole, orciprenaline, pirbuterol, procaterol, (R,R)-formoterol, reproterol, ritodrine, rimoterol, salbutamol, salmeterol, sibenadet, sulfonterol, terbutaline, tulobuterol, GSK-597901, milveterol, abediterol (LAS100977), KUL-1248, carmoterol and indacaterol optionally in the form of their racemates, their enantiomers, their diastereomers, and mixtures thereof, and optionally their pharmacologically-compatible acid addition salts.

Since aclidinium has a long duration of action, it is preferred that it is combined with long-acting β2-agonists (also known as LABAs). The combined drugs could thus be administered once or twice a day.

Particularly preferred LABAs are formoterol, salmeterol and GSK-597901, milveterol, LAS100977 (5-(2-{[6-(2,2-difluoro-2-phenylethoxy)hexyl]amino}-1(R)-hydroxyethyl)-8-hydroxyquinolin-2(1H)-one), KUL-1248, carmoterol and indacaterol optionally in the form of their racemates, their enantiomers, their diastereomers and mixtures thereof, and optionally their pharmacologically-compatible acid addition salts. More preferred are salmeterol, formoterol, abediterol (LAS100977), and indacaterol. Still more preferred are salmeterol, formoterol and LAS100977 (5-(2-{[6-(2,2-difluoro-2-phenylethoxy)hexyl]amino}-1(R)-hydroxyethyl)-8-hydroxyquinolin-2(1H)-one), in particular salmeterol xinafoate, formoterol fumarate and LAS100977 (5-(2-{[6-(2,2-difluoro-2-phenylethoxy)hexyl]amino}-1(R)-hydroxyethyl)-8-hydroxyquinolin-2(1H)-one).

For example, the invention provides a pharmaceutical composition for inhalation comprising aclidinium in the form of a dry powder of a pharmaceutically acceptable salt, e.g., bromide, in admixture with lactose particles, together with formoterol fumarate, (i) providing a delivered dose of aclidinium equivalent to about 322 μg aclidinium free base and/or a fine particle dose equivalent to about 140 μg aclidinium bromide together with a single metered nominal dose of about 5-25 μg (e.g. 6, 8.5, 12, 18 or 24 μg, for example 6 or 12 μg) formoterol fumarate or (ii) in a multidose dry powder inhaler device calibrated to provide a delivered dose of aclidinium equivalent to about 322 μg aclidinium free base and/or a fine particle dose equivalent to about 140 μg aclidinium bromide together with a metered nominal dose of about 5-25 μg (e.g. 6, 8.5, 12, 18 or 24 μg, for example 6 or 12 μg) formoterol fumarate. A metered nominal dose of about 6 μg formoterol fumarate typically corresponds to a delivered dose of about 4.5 μg formoterol fumarate and a metered nominal dose of about 12 μg formoterol fumarate typically corresponds to a delivered dose of about 9 μg formoterol fumarate.

By a delivered dose of “about 4.5 μg formoterol fumarate” it is meant a target dose of 4.5 μg formoterol fumarate subject to variation within the normal limits of acceptance for the dispensing system, e.g. 2.9-6.1 μg formoterol fumarate (plus/minus 35%, acceptable variations as defined by the European and US Pharmacopeia) or preferably 3.3-5.6 μg formoterol fumarate (plus/minus 25%, acceptable variations as defined by the current most stringent requirement, the US FDA draft guidance for inhaler), or more preferably 3.8-5.2 μg formoterol fumarate (plus/minus 15%, acceptable variations defined by the CHMP Guideline on the Pharmaceutical Quality of Inhalation and Nasal Products) or especially 4.0-5.0 μg formoterol fumarate (or within the metered dosing accuracy of the inhaler).

By a delivered dose of “about 9 μg formoterol fumarate” it is meant a target dose of 9 μg formoterol fumarate subject to variation within the normal limits of acceptance for the dispensing system, e.g. 5.8-12.2 μg formoterol fumarate (plus/minus 35%, acceptable variations as defined by the European and US Pharmacopeia) or preferably 6.7-11.3 μg formoterol fumarate (plus/minus 25%, acceptable variations as defined by the current most stringent requirement, the US FDA draft guidance for inhaler), or more preferably 7.6-10.3 μg formoterol fumarate (plus/minus 15%, acceptable variations defined by the CHMP Guideline on the Pharmaceutical Quality of Inhalation and Nasal Products) or especially 8.1-9.9 μg formoterol fumarate (or within the metered dosing accuracy of the inhaler).

In a particular embodiment, a metered nominal dose of about 6 μg formoterol fumarate typically corresponds to a delivered dose of about 5.8 μg formoterol fumarate.

By a delivered dose of “about 5.8 μg formoterol fumarate” it is meant a target dose of 5.8 μg formoterol fumarate subject to variation within the normal limits of acceptance for the dispensing system, e.g. 3.7-7.8 μg formoterol fumarate (plus/minus 35%, acceptable variations as defined by the European and US Pharmacopeia) or preferably 4.3-7.3 μg formoterol fumarate (plus/minus 25%, acceptable variations as defined by the current most stringent requirement, the US FDA draft guidance for inhaler), or more preferably 4.9-6.6 μg formoterol fumarate (plus/minus 15%, acceptable variations defined by the CHMP Guideline on the Pharmaceutical Quality of Inhalation and Nasal Products) or especially 5.2-6.4 μg formoterol fumarate (or within the metered dosing accuracy of the inhaler).

In another particular embodiment, a metered nominal dose of about 12 μg formoterol fumarate typically corresponds to a delivered dose of about 11.8 μg formoterol fumarate.

By a delivered dose of “about 11.8 μg formoterol fumarate” it is meant a target dose of 11.8 μg formoterol fumarate subject to variation within the normal limits of acceptance for the dispensing system, e.g. 7.6-15.9 μg formoterol fumarate (plus/minus 35%, acceptable variations as defined by the European and US Pharmacopeia) or preferably 8.8-14.8 μg formoterol fumarate (plus/minus 25%, acceptable variations as defined by the current most stringent requirement, the US FDA draft guidance for inhaler), or more preferably 10.0-13.6 μg formoterol fumarate (plus/minus 15%, acceptable variations defined by the CHMP Guideline on the Pharmaceutical Quality of Inhalation and Nasal Products) or especially 10.6-13.0 μg formoterol fumarate (or within the metered dosing accuracy of the inhaler).

In another particular embodiment, a metered nominal dose of about 12 μg formoterol fumarate typically corresponds to a delivered dose of about 12 μg formoterol fumarate.

By a delivered dose of “about 12 μg formoterol fumarate” it is meant a target dose of 12 μg formoterol fumarate subject to variation within the normal limits of acceptance for the dispensing system, e.g. 7.8-16.2 μg formoterol fumarate (plus/minus 35%, acceptable variations as defined by the European and US Pharmacopeia) or preferably 9.0-15.0 μg formoterol fumarate (plus/minus 25%, acceptable variations as defined by the current most stringent requirement, the US FDA draft guidance for inhaler), or more preferably 10.2-13.8 μg formoterol fumarate (plus/minus 15%, acceptable variations defined by the CHMP Guideline on the Pharmaceutical Quality of Inhalation and Nasal Products) or especially 10.8-13.2 μg formoterol fumarate (or within the metered dosing accuracy of the inhaler).

The pharmaceutical composition for inhalation comprising aclidinium and a 02-agonist, for example, formoterol or abediterol (LAS100977), can be administered one or more times per day. Preferably once or twice a day.

Examples of suitable PDE4 inhibitors that can be combined with aclidinium in the present invention are benafentrine dimaleate, etazolate, denbufylline, rolipram, cipamfylline, zardaverine, arofylline, filaminast, tipelukast, tofimilast, piclamilast, tolafentrine, mesopram, drotaverine hydrochloride, lirimilast, roflumilast, cilomilast, oglemilast, apremilast, 6-[2-(3,4-Diethoxyphenyl)thiazol-4-yl]pyridine-2-carboxylic acid (tetomilast), (R)-(+)-4-[2-(3-Cyclopentyloxy-4-methoxyphenyl)-2-phenylethyl]pyridine (CDP-840), N-(3,5-Dichloro-4-pyridinyl)-2-[1-(4-fluorobenzyl)-5-hydroxy-1H-indol-3-yl]-2-oxoacetamide (GSK-842470), 9-(2-Fluorobenzyl)-N6-methyl-2-(trifluoromethyl)adenine (NCS-613), N-(3,5-Dichloro-4-pyridinyl)-8-methoxyquinoline-5-carboxamide (D-4418), N-[9-Methyl-4-oxo-1-phenyl-3,4,6,7-tetrahydropyrrolo[3,2,1-jk][1,4]benzodiazepin-3(R)-yl]pyridine-4-carboxamide, 3-[3-(Cyclopentyloxy)-4-methoxybenzyl]-6-(ethylamino)-8-isopropyl-3H-purine hydrochloride (V-11294A), 6-[3-(N,N-Dimethylcarbamoyl)phenylsulfonyl]-4-(3-methoxyphenylamino)-8-methylquinoline-3-carboxamide hydrochloride (GSK-256066), 4-[6,7-Diethoxy-2,3-bis(hydroxymethyl)naphthalen-1-yl]-1-(2-methoxyethyl)pyridin-2(1H)-one (T-440), (−)-trans-2-[3′-[3-(N-Cyclopropylcarbamoyl)-4-oxo-1,4-dihydro-1,8-naphthyridin-1-yl]-3-fluorobiphenyl-4-yl]cyclopropanecarboxylic acid (MK-0873), CDC-801, UK-500001, BLX-914, 2-carbomethoxy-4-cyano-4-(3-cyclopropylmethoxy-4-difluroromethoxyphenyl)cyclohexan-one, cis [4-cyano-4-(3-cyclopropylmethoxy-4-difluoromethoxyphenyl)cyclohexan-1-ol, 5(S)-[3-(Cyclopentyloxy)-4-methoxyphenyl]-3(S)-(3-methylbenzyl)piperidin-2-one (IPL-455903), ONO-6126 (Eur Respir J 2003, 22(Suppl. 45): Abst 2557) and the compounds claimed in the PCT patent applications number WO 03/097613, WO 2004/058729, WO 2005/049581, WO 2005/123693 and WO 2005/123692.

Examples of suitable corticosteroids and glucocorticoids that can be combined with aclidinium in the present invention are prednisolone, methylprednisolone, dexamethasone, dexamethasone cipecilate, naflocort, deflazacort, halopredone acetate, budesonide, beclomethasone dipropionate, hydrocortisone, triamcinolone acetonide, fluocinolone acetonide, fluocinonide, clocortolone pivalate, methylprednisolone aceponate, dexamethasone palmitoate, tipredane, hydrocortisone aceponate, prednicarbate, alclometasone dipropionate, Butixocort propionate, RPR-106541, halometasone, methylprednisolone suleptanate, mometasone furoate, rimexolone, prednisolone farnesylate, ciclesonide, deprodone propionate, fluticasone propionate, fluticasone furoate, halobetasol propionate, loteprednol etabonate, betamethasone butyrate propionate, flunisolide, prednisone, dexamethasone sodium phosphate, triamcinolone, betamethasone 17-valerate, betamethasone, betamethasone dipropionate, 21-Chloro-11beta-hydroxy-17alpha-[2-(methylsulfanyl)acetoxy]-4-pregnene-3,20-dione, Desisobutyrylciclesonide, hydrocortisone acetate, hydrocortisone sodium succinate, NS-126, prednisolone sodium phosphate, hydrocortisone probutate, prednisolone sodium metasulfobenzoate and clobetasol propionate, especially budesonide or mometasone.

For example, the invention provides a pharmaceutical composition for inhalation comprising aclidinium in the form of a dry powder of a pharmaceutically acceptable salt, e.g., bromide, in admixture with a pharmaceutically acceptable carrier, e.g., lactose particles, together with mometasone furoate, (i) providing a delivered dose of aclidinium equivalent to about 322 μg aclidinium free base and/or a fine particle dose equivalent to about 140 μg aclidinium bromide together with a single metered nominal dose of about 100-900 μg (e.g., 100, 110, 200, 220, 300, 330, 400, 440, 800 or 880 μg, for example 200-450, e.g. 220 or 440 μg) mometasone furoate, or (ii) in a multidose dry powder inhaler device calibrated to provide a delivered dose of aclidinium equivalent to about 322 μg aclidinium free base and/or a fine particle dose equivalent to about 140 μg aclidinium bromide together with a metered nominal dose of about 100-900 μg (e.g. 100, 110, 200, 220, 300, 330, 400, 440, 800 or 880 μg, for example 200-450, e.g. 220 or 440 μg) mometasone furoate.

The pharmaceutical composition for inhalation comprising aclidinium and a corticosteroid, for example mometasone furoate, can be administered one or more times per a day. Preferably once or twice a day.

The invention also provides a pharmaceutical composition comprising aclidinium, a β2-agonist as defined above and a corticosteroid, as defined above. Most preferred β2-agonists are selected from abediterol (LAS100977) and formoterol. Most preferred corticosteroid is mometasone furoate. These triple combinations are suitable for administration once or twice a day.

The following examples are given in order to provide a person skilled in the art with a sufficiently clear and complete explanation of the present invention, but should not be considered as limiting of the essential aspects of its subject, as set out in the preceding portions of this description.

### EXAMPLES

### Example 1

### 1.1. Pharmaceutical Composition for Inhalation Comprising Aclidinium Bromide and Lactose

A pharmaceutical composition in a batch size of 80 kg comprising aclidinium bromide and alpha-lactose monohydrate having a d10 of 90-160 μm, a d50 of 170-270 μm and a d90 of 290-400 μm, was prepared.

Aclidinium bromide (2.462 Kg) and alpha-lactose monohydrate (77.538 Kg) were blended in a Bohle blender, the mixture was sieved through a sieving-machine Bohle BTS and finally, the mixture was blended in a Bohle blender.

Genuair® (H. Chrystyn et al. (2009)) cartridges were filled with the composition. The cartridges were calibrated to provide 30 or 60 metered doses. Each actuation of the Genuair® provided a metered dose of 13 mg of the composition described above.

### 1.2. Measurement of the Delivered Dose

The measurement of the delivered dose (amount of the drug which is available at the mouth for inhalation) of the pharmaceutical composition described in point 1.1. is carried out based on European Pharmacopoiea¹ 7th Edition (7.0), Chapter 2.9.18 and US Pharmacopoiea² USP36-NF31, Chapter 601; using a “Collection Tube” apparatus (CT). For this, the Genuair® inhaler is fitted to the Collection Tube via an adapter, the dosage key of the Genuair® inhaler is pressed and released and then 2 L or 4 L of air are sucked through the inhaler (inspiratory flow rate through the inhaler was approx. 65 L/min at a pressure drop of 4 KPa) and the Collection Tube. Subsequently, the inhalation powder delivered to the Collection Tube is extracted with solvent and analyzed using High Performance Liquid chromatography equipment (HPLC).

The mean delivered dose per actuation (per inhalation) was 322 μg aclidinium (aclidinium free ammonium cation), which corresponds to 375 μg aclidinium bromide. The accepted variance defined by the CHMP Guideline³ on the Pharmaceutical Quality on Inhalation and Nasal Products was 274-370 μg aclidinium (aclidinium free ammonium cation), which corresponds to 319-431 μg aclidinium bromide.

### Example 1.3. Measurement of the Fine Particle Dose (FPD)

The test on the aerodynamic assessment of the fine particles (FPD<5 μm) of the inhalation powder composition is carried out in the Genuair® inhaler. The fine particle dose of the pharmaceutical composition described in point 1.1. was calculated on basis of the principles of the aerodynamic assessment of fine particles according to the European Pharmacopoiea¹ 7th Edition (7.0), Chapter 2.9.18 and US Pharmacopoiea² USP36-NF31, Chapter 601; by the aid of aerodynamic impactor analyses using a modified Andersen Cascade Impactor (ACI), 60 L/min-configuration including pre-separator, stage −1, −0, and stage 1-7 (filter stage). The content of the active ingredient on each stage of the impactor is determined my means of HPLC.

The fine particle dose (FPD<5 μm) was calculated according to European Pharmacopoiea¹ 7th Edition (7.0), Chapter 2.9.18 and US Pharmacopoiea² USP36-NF31, Chapter 601; by point to point interpolation per dosage. Linear point to point interpolation is done between the stages with a corresponding effective cut-off diameter which enclose the 5 μm mark

To obtain the fine particle dose, the cumulative percent value (y-value) at which the line of data plot crosses 5 μm mark is determined. The found cumulative percent must be multiplied by the sum of mass of the active ingredient per dosage on stage −1-stage 7 (Filter) to obtain the fine particle dose, <5 μm, in μg.

FPD [μg]=yFPD*F/100%

FPD=Fine particle dose<5 μm of the active ingredient per dosage [μg].
yFPD=y-value of cumulative percentage of mass at a particle size of 5 μm evaluated by linear point to point interpolation [%].
F=sum of mass on stage −1-stage 7 (filter) per dosage [μg].

The mean fine particle dose per actuation (per inhalation) was 120 μg aclidinium (aclidinium free ammonium cation), which corresponds to 140 μg aclidinium bromide. The accepted variance was 86-163 μg aclidinium (aclidinium free ammonium cation), which corresponds to 100-190 μg aclidinium bromide
[1] United States Pharmacopeial Convention. Chapter 601. Aerosols, metered-dose inhalers, and dry powder inhalers. In: USP36-NF31. Rockville, Md.: USP; 2013:242-262
[2] European Pharmacopeia. Section 2.9.18—Preparations for inhalation: Aerodynamic assessment of fine particles, 7th Edition (7.0), Council of Europe, Strasbourg, 2010, pp 274-285
[3] Committee for Medicinal Products for Human Use (CHMP). Guideline on the Pharmaceutical Quality of Inhalation and Nasal Products. Doc. Ref. EMEA/CHMP/QWP/49313/2005 Corr, 2006

Modifications, which do not affect, alter, change or modify the essential aspects of the pharmaceutical compositions described, are included within the scope of the present invention.

1-26. (canceled)

27. A dry powder pharmaceutical inhalation composition comprising aclidinium or a pharmaceutically acceptable salt thereof in admixture with a suitable carrier, wherein the pharmaceutical composition provides a) a mean delivered dose of aclidinium equivalent to 322 micrograms aclidinium free base, or b) a mean fine particle dose of aclidinium equivalent to 140 micrograms aclidinium bromide.

28. The pharmaceutical composition according to claim 27, in the form of a single-dose dry powder formulation comprising a) a mean delivered dose of aclidinium equivalent to 322 micrograms aclidinium free base, or b) a mean fine particle dose equivalent to 140 micrograms aclidinium bromide.

29. The pharmaceutical composition according to claim 27, in the form of a multi-dose dry powder formulation for administration in a multidose dry powder inhaler device calibrated to provide a) a mean delivered dose of aclidinium equivalent to 322 micrograms aclidinium free base, or b) a mean fine particle dose equivalent to 140 micrograms aclidinium bromide.

30. The pharmaceutical composition according to claim 27, wherein the pharmaceutically acceptable salt of aclidinium is aclidinium bromide.

31. The pharmaceutical composition according to claim 27, wherein the carrier is lactose in the form of alpha-lactose monohydrate.

32. The pharmaceutical composition according to claim 27, wherein the ratio of aclidinium to the carrier ranges from 1:25 to 1:75 by weight.

33. The pharmaceutical composition according to claim 27, wherein the average particle diameter of aclidinium ranges from 2 μm to 5 μm.

34. The pharmaceutical composition according to claim 31, wherein the lactose comprises particles having a d10 ranging from 90 μm to 160 μm, a d50 ranging from 170 μm to 270 μm, and a d90 ranging from 290 μm to 400 μm.

35. The pharmaceutical composition according to claim 27, further comprising an effective amount of at least one additional active agent chosen from β2-agonists, PDE IV inhibitors, or corticosteroids.

36. The pharmaceutical composition according to claim 35, wherein the at least one additional active agent is chosen from fluticasone propionate, fluticasone furoate, formoterol, salmeterol, budesonide, or mometasone, and wherein the at least one additional active agent is in free or pharmaceutically acceptable salt form.

37. The pharmaceutical composition according to claim 36, wherein the at least one additional active agent is formoterol fumarate in an amount ranging from about 5 micrograms to 25 micrograms per metered nominal dose.

38. The pharmaceutical composition according to claim 37, wherein the at least one additional active agent is formoterol fumarate in an amount of about 6 micrograms per metered nominal dose.

39. The pharmaceutical composition according to claim 37, wherein the at least one additional active agent is formoterol fumarate in an amount of about 12 micrograms per metered nominal dose.

40. A method of treating a respiratory condition chosen from asthma or chronic obstructive pulmonary disease, comprising administering a) a mean delivered dose of aclidinium equivalent to 322 micrograms of aclidinium free base, or b) a mean fine particle dose equivalent to 140 micrograms aclidinium bromide by inhalation to a patient in need of such treatment.

41. A method of treating a respiratory condition chosen from asthma or chronic obstructive pulmonary disease, comprising administering twice daily a) a mean delivered dose of aclidinium equivalent to 322 micrograms of aclidinium free base, or b) a fine particle dose equivalent to 140 micrograms aclidinium bromide by inhalation to a patient in need of such treatment.

42. The method according to claim 40, comprising administering a dry powder pharmaceutical composition comprising aclidinium or a pharmaceutically acceptable salt thereof in admixture with a suitable carrier, wherein the pharmaceutical composition is administered in a mean delivered dose of aclidinium equivalent to 322 micrograms aclidinium free base, or b) a mean fine particle dose equivalent to 140 micrograms aclidinium bromide by inhalation to a patient in need of such treatment.

43. The method according to claim 42, further comprising administering an effective amount of at least one additional active agent chosen from β2-agonists, PDE IV inhibitors, or corticosteroids.

44. The method according to claim 43, wherein the at least one additional active agent is chosen from fluticasone propionate, fluticasone furoate, formoterol, salmeterol, budesonide, or mometasone, wherein the at least one additional active agent is in free or pharmaceutically acceptable salt form.

45. The method according to claim 44, wherein the at least one additional active agent is formoterol fumarate in an amount ranging from about 5 micrograms to 25 micrograms per metered nominal dose.

46. A dry powder inhaler device calibrated to deliver, upon actuation, a) a mean delivered dose of aclidinium equivalent to 322 micrograms aclidinium free base, or b) a mean fine particle dose equivalent to 140 micrograms aclidinium bromide.

47. The dry powder inhaler device according to claim 46, wherein the device is single-dose.

48. The pharmaceutical composition according to claim 27, wherein the ratio of aclidinium to the carrier ranges from 1:25 to 1:50 by weight.

49. The dry powder inhaler device according to claim 46, wherein the device is multi-dose.

50. The method according to claim 42, wherein the pharmaceutically acceptable salt of aclidinium is aclidinium bromide.

51. The method according to claim 42, wherein the average particle diameter of aclidinium ranges from 2 μm to 5 μm.

52. The method according to claim 42, wherein the carrier is lactose in the form of alpha-lactose monohydrate.

53. The method according to claim 52, wherein the lactose comprises particles having a d10 ranging from 90 μm to 160 μm, a d50 ranging from 170 μm to 270 μm, and a d90 ranging from 290 μm to 400 μm.

54. The method according to claim 42, wherein the ratio of aclidinium to the carrier ranges from 1:25 to 1:50 by weight.
